(19) United States
(12) Patent Application Publication
(10) Pub. No.: US 2012/0203107 A1
(43) Pub. Date: Aug. 9, 2012

(54) ULTRASOUND MEASURING APPARATUS AND CONTROL METHOD THEREOF

(75) Inventor: Dong Hwan KIM, Gyeonggi-do (KR)

(73) Assignee: SAMSUNG ELECTRONICS CO., LTD., Gyeonggi-Do (KR)

(21) Appl. No.: 13/366,620

(22) Filed: Feb. 6, 2012

(30) Foreign Application Priority Data
Feb. 7, 2011 (KR) 10-2011-0010427

(51) Int. Cl. A61B 8/00

(52) U.S. Cl. 600/443

(57) ABSTRACT

Provided are an ultrasound measuring device that uses multiple ultrasound probes to acquire a plurality of images and synthesizes the same to produce a synthesized image of a subject, as well as a control method thereof. The ultrasound measuring device includes: multiple ultrasound probes having sensors to detect relative positions between the probes; and a control part that uses a plurality of signals transmitted from the multiple ultrasound probes to generate a plurality of image signals and uses position information between respective pairs of the multiple ultrasound probes, which position information is transmitted from the corresponding sensors, to compensate for an error relating to the corresponding image signals from the plurality of image signals.

### CLAIM OF PRIORITY

This application claims priority from Korean Patent Application No. 10-2011-0010427, filed on Feb. 7, 2011, in the Korean Intellectual Property Office, the disclosure of which is incorporated herein by reference in its entirety.

### BACKGROUND

1. Field

Exemplary embodiments of the present disclosure relate to an ultrasound measuring apparatus for inspecting an object using multiple ultrasound probes and a control method thereof.

2. Description of the Related Art

An ultrasound measuring apparatus has advantages, such as a small size, indication of measured results in real time, and no radiation exposure, as compared with other measurement devices, thus exhibiting high stability.

Accordingly, an ultrasound measuring apparatus is typically widely used together with an imaging diagnosis apparatus such as, for example, an X-ray device, a CT scanner, an MRI device, etc.

Such an ultrasound measuring apparatus may deliver an ultrasound signal from the surface of a subject to be measured (i.e., “measured subject”) toward a measuring site, receive an ultrasound signal reflected from an internal tissue of the measured subject, and then use information included in the received signal to obtain tomographic images or blood flow images of the internal tissue of the measured subject.

The ultrasound measuring apparatus utilizes the Doppler effect to obtain information relating to blood flow. A method for measuring blood flow using the Doppler effect can include non-invasively measuring a blood flow rate in real time, thus being widely applicable for purposes relating to non-invasive and real time measurement.

The ultrasound measuring apparatus using the Doppler effect transmits an ultrasound signal from an ultrasound probe toward a subject to be measured, receives an ultrasound signal reflected from a target in the measured subject, and measures a frequency variation of the received ultrasound signal caused by movement of the target, thus indicating a velocity of the target.

That is, when a target moves, a central frequency of a reflected ultrasound signal may be varied from a central frequency of the ultrasound signal transmitted to a measured subject and, based on an extent of variation, a velocity of a target in the measured subject may be calculated.

As described above, in the case in which a blood flow rate is measured by using the Doppler effect, a measuring direction of the measuring apparatus is usually oriented to an expansion direction of the blood flow at a predetermined angle. In particular, as a measuring angle approaches a right angle, a problem such as occurrence of error in measuring the blood flow rate and/or direction may often be encountered.

### SUMMARY

According to aspects of exemplary embodiments of the present disclosure, there are provided an ultrasound measuring apparatus that acquires a plurality of images using multiple ultrasound probes and synthesizes the same, in turn producing images of a subject, as well as a control method thereof.

In one aspect, an ultrasound measuring apparatus according to an exemplary embodiment of the present disclosure includes: multiple ultrasound probes having sensors capable of detecting relative positions; a control part that uses a plurality of signals transmitted from the multiple ultrasound probes to generate a plurality of image signals, and uses position information between respective pairs of the multiple ultrasound probes, which position information is transmitted from the corresponding sensors, to compensate for errors relating to the corresponding image signals from the plurality of image signals.

A respective sensor may inspect position information including, for example, an angle and/or distance between multiple ultrasound probes.

With reference to a position of one of the ultrasound probes, relative positions of one or more other ultrasound probes may be determined as the position information.

The control part may produce a synthesized image signal by synthesizing plural image signals after compensating for errors arising therefrom.

In another aspect, a controlling method of an ultrasound measuring apparatus according to another exemplary embodiment of the present disclosure includes: receiving signals from multiple ultrasound probes having respective sensors that can inspect relative positions between the probes; generating a plurality of image signals using the received signals from the multiple ultrasound probes; and, after compensating for errors relating to the plurality of image signals, synthesizing two or more of the compensated signals to produce a synthesized image signal.

The reception of signals from the multiple ultrasound probes having respective sensors may comprise receiving an electrical signal generated by detecting a subject from different positions using the multiple ultrasound probes, and using position information, including an angle and/or distance between individual ultrasound probes, detected using the respective sensors.

With reference to a position of one of the ultrasound probes, relative positions of other ultrasound probes may be determined as the position information.

The generation of the plurality of image signals using signals received from the multiple ultrasound probes may comprise inspecting a subject by each of the multiple ultrasound probes from different respective positions to obtain a respective signal, and producing a plurality of image signals of the subject by using the obtained signals.

The production of a synthesized image signal by compensating for an error relating to the plurality of image signals and then processing two or more of the compensated image signals into a synthesized image signal may include: using relative position information between two or more of the multiple ultrasound probes determined from signals transmitted from the respective sensors to compensate for an error arising between two or more of the plurality of image signals; and processing the compensated image signals into the synthesized image signal.

The compensation of the error arising between the two or more of the plurality of image signals may comprise compensating for an artifact, which may occur in each of the image signals due to different inspection sites, using relative position information between two or more of the multiple ultrasound probes determined from measurements performed by the sensor.

In yet another aspect, in accordance with an exemplary embodiment of the present disclosure, a plurality of images may be acquired simultaneously from different angles relative to a subject and, by comparing such images and removing an artifact possibly occurring in the image, more distinct images may be acquired.

Moreover, blood flow information may be more clearly and accurately provided.

### BRIEF DESCRIPTION OF THE DRAWINGS

These and/or other aspects of the disclosure will become apparent and more readily appreciated from the following description of exemplary embodiments, taken in conjunction with the accompanying drawings, of which:

FIG. 1 is a block diagram illustrating a configuration of an ultrasound measuring apparatus according to an exemplary embodiment of the present disclosure;

FIG. 2 illustrates a measurement of a blood flow rate using an ultrasound measuring apparatus according to an exemplary embodiment of the present disclosure; and

FIG. 3 illustrates a control method of an ultrasound measuring apparatus according to an exemplary embodiment of the present disclosure.

### DETAILED DESCRIPTION

Hereinafter, advantageous features and characteristics of the present disclosure and practical methods thereof will be clearly understood through the following detailed description of exemplary and illustrative embodiments with reference to the accompanying drawings. However, at least one exemplary embodiment of the present disclosure may be embodied in various other forms, which are not particularly restricted to those described herein.

In the accompanying drawings, like reference numerals denote elements having substantially the same configurations or performing similar functions and actions throughout the drawings.

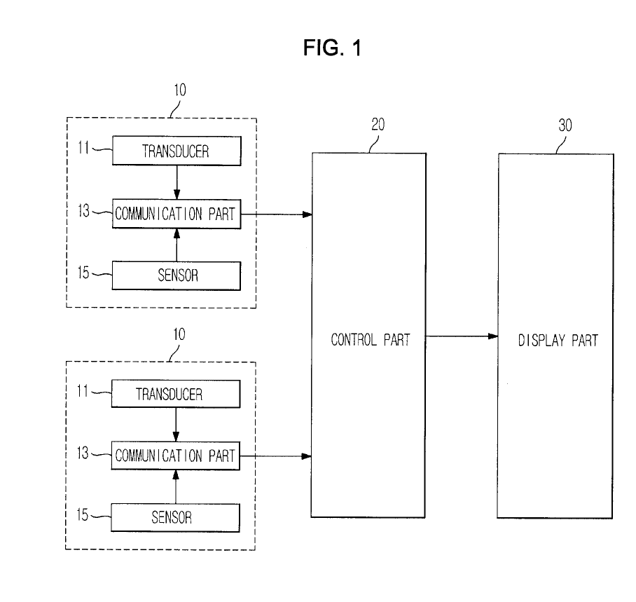

FIG. 1 is a block diagram illustrating a construction of an ultrasound measuring apparatus according to an exemplary embodiment of the present disclosure.

The ultrasound measuring apparatus according to one embodiment of the present disclosure comprises: multiple ultrasound probes 10, each of which irradiates ultrasound toward a subject and receives a reflected ultrasound signal, thus generating an electrical signal; a control part 20 that accepts the electrical signal relating to the subject transmitted from each of the multiple ultrasound probes 10 and generates an image signal based on the electrical signals; and a display part 30 that receives the image signal from the control part 20 and displays an image of the inside of the subject.

Each of the multiple ultrasound probes may include a transducer 11 to generate ultrasound, a sensor 15 to detect position information relating to the multiple ultrasound probes, and a communication part 13 to transmit the signal received from the transducer 11 and the sensor 15 toward the control part 20.

The transducer 11 may generate and irradiate ultrasound toward a subject, while receiving an ultrasound signal reflected from a target inside the subject and converting the same into an electrical signal. The transducer 11 uses a piezoelectric element to produce ultrasound and receives an ultrasound signal reflected from a target inside the subject, in turn converting the same into an electrical signal.

The piezoelectric element is a material exhibiting piezoelectric effects, wherein a voltage is generated by applying mechanical pressure, and mechanical deformation may be caused by applying the voltage. That is, the piezoelectric element is a material that converts electrical energy into mechanical vibration energy or mechanical vibration energy into electrical energy. Accordingly, in the case where a voltage is applied to the piezoelectric element, the piezoelectric element causes mechanical vibration, in turn generating ultrasound. Alternatively, when the piezoelectric element receives an ultrasound signal, mechanical vibration energy may be converted into an electrical signal. Such a piezoelectric element may be formed using one or more of the following materials: zirconic titanate (PZT) ceramics, PZMT single crystals made of a solid solution comprising lead magnesium niobate and lead titanate; or PZNT single crystals made of a solid solution comprising lead zinc niobate and lead titanate, without being particularly limited thereto.

A sensor 15 is mounted on each of the multiple ultrasound probes 10 and, when one of the multiple ultrasound probes 10 measures a subject, the respective sensor 15 may sense a relative position between two of the multiple ultrasound probes 10. For instance, if two ultrasound probes 10 are used to determine the ultrasound of a subject, with reference to a first sensor 15 mounted on a first ultrasound probe 10, a second sensor 15 mounted on a second ultrasound probe 10 may sense a relative position of the second ultrasound probe 10 with respect to the first ultrasound probe 10. The relative position of the second ultrasound probe may include information as to how far the second ultrasound probe is spaced apart from the first ultrasound probe when the measurement is implemented, and/or an angle between the second ultrasound probe and the first ultrasound probe when measurement is implemented. Briefly, information relating to a distance and an angle between the first and second ultrasound probes may be included. In this regard, the angle between the first and second ultrasound probes may be an angle formed between an expansion side of a face of the first ultrasound probe adjacent to the subject and an expansion side of a face of the second ultrasound probe adjacent to the subject, without being particularly limited thereto.

As described above, the method of sensing a position between the multiple ultrasound probes 10 is only an exemplary embodiment. However, other methods for sensing a position between the multiple ultrasound probes 10 using sensors 15 may be used.

The communication part 13 may receive an electrical signal generated as a result of measuring the subject from the transducer 11 and information relating to relative positions between the multiple ultrasound probes 10 sensed by the sensors 15, and then transmit the received signals to the control part 20. Communication between the communication part 13 and the control part 20 may be executed in either a wired or wireless mode.

The control part 20 may receive the electrical signal generated as a result of measuring the subject from the communication part 13, as well as a signal containing the position information relating to the multiple ultrasound probes 10.

The control part 20 may convert a plurality of electrical signals received from the multiple ultrasound probes 10 into image signals, thus enabling an internal image of the subject to be displayed on a display 30.

For instance, by receiving two different electrical signals from two (first and second) ultrasound probes 10, first and second image signals may be produced. Because the first and second ultrasound probes are spaced apart from each other and each irradiates ultrasound to the subject from a different angle or direction, the first and second image signals may display respective images corresponding to different angles relative to the inside of the subject.

The control part 20 may use the position information relating to the multiple ultrasound probes 10 sensed by the sensor 15 to execute a compensation of an error relating to the corresponding image signals, before synthesizing the corresponding image signals and producing a synthesized image signal.

For instance, because the respective image signals exhibit measured results of the subject from different angles and/or positions, a tissue having high density may be present in an image detected from a first measuring direction, which therefore does not show an image of the rear side of the tissue, while the image of the rear side of the high density tissue may be displayed in an image detected from another measuring direction (i.e., a measuring direction which is different from the first measuring direction). That is, when measuring the subject from different angles, an image not shown from any one measuring result may be displayed from the other measuring result.

If desired images include not only a front image of a tissue having high density, but also a rear image thereof, the control part 20 uses position information relating to multiple ultrasound probes 10, as sensed by the sensors 15, and compares a plurality of image signals, thus compensating for an error relating to the respective image signals, and then produces a synthesized image signal to display desired images.

Such a method may also be employed in measuring blood flow rate. For a measurement of blood flow rate, in the case where only one ultrasound probe 10 is used, the error in the measured result may be increased if a blood flow direction is oriented at a predetermined angle relative to an ultrasound proceeding direction, especially if the predetermined angle is nearly equal to a right angle.

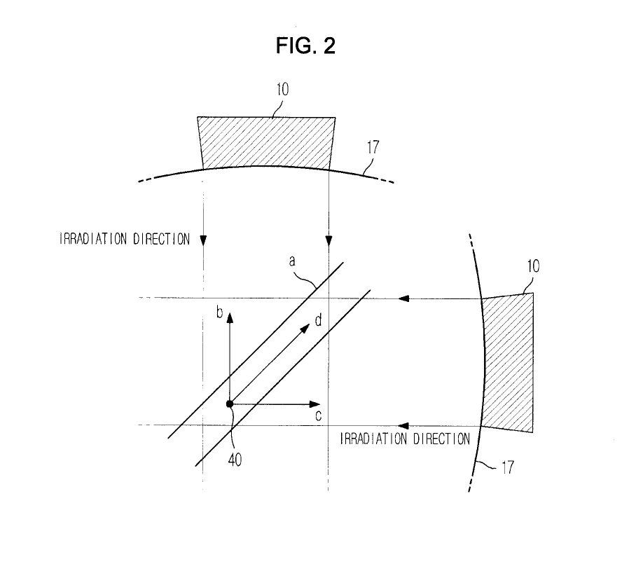

However, as illustrated in FIG. 2, in the case where two ultrasound probes 10 are used for a measurement to exhibit a velocity of the ultrasound reflecting from blood cells 40 and then returning, a blood flow rate may be calculated as a vector sum. That is, the control part 20 may receive two electrical signals from two respective ultrasound probes 10 and, using the relative position information with respect to the two ultrasound probes 10 sensed by the sensors 15, the received electrical signals are compared, compensated and synthesized, thus enabling calculation of a more accurate blood flow rate and displaying the calculated result as an image.

The display part 30 may receive the synthesized image signal obtained by compensating and synthesizing the two image signals in the control part 20 and, according to the synthesized image signal, an internal image of the subject may be displayed.

FIG. 2 illustrates a measurement of a blood flow rate through an ultrasound measuring apparatus according to an exemplary embodiment of the present inventive concept.

Two ultrasound probes 10 may contact a subject 17 from different angles and irradiate ultrasound. The irradiated ultrasound passes through a vesicular tract (labeled “a”) of the subject 17 and, in such a process, the ultrasound collides with blood cells 40 in blood fluid flowing in the vesicular tract “a”, and then the ultrasound is reflected.

The ultrasound probe 10 may receive the reflected ultrasound signal, convert it into an electrical signal and transfer the electrical signal to the control part 20. The control part 20 may also receive relative position information relating to two ultrasound probes 10 sensed by the corresponding sensors 15 of the respective ultrasound probes 10, in addition to receiving the electrical signal from each of the two ultrasound probes 10.

The control part 20 may use the relative position information relating to the ultrasound probes to calculate velocity vectors “b” and “c” of the ultrasound from the electrical signal, and calculate a blood flow rate “d” by computing a vector sum of the velocity vectors “b” and “c”.

In addition, by converting each electrical signal into an image signal and, as described above, using the relative position information between the ultrasound probes to compensate for an error relating to the image signals, a synthesized image signal displaying an image without any artifacts may be successfully synthesized. The display part 30 may receive the synthesized image signal from the control part 20 and use the synthesized image signal to display the flow of a blood fluid as an image.

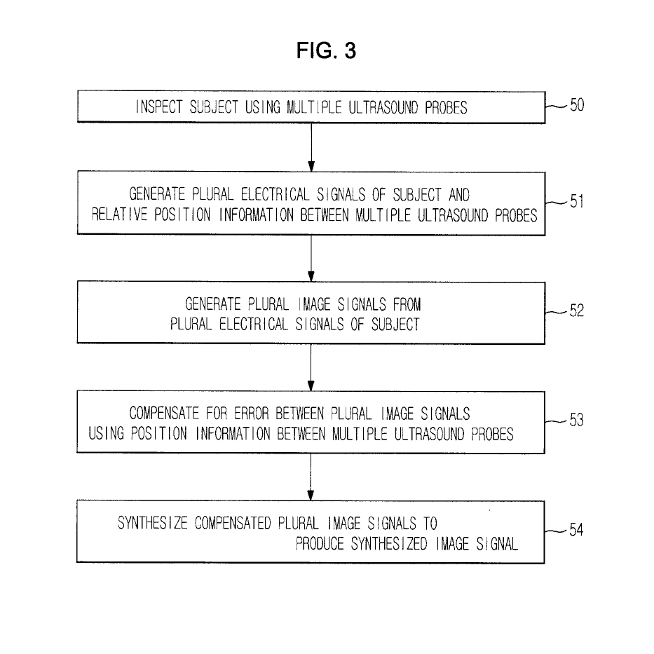

FIG. 3 is a flow diagram illustrating a control method of an ultrasound measuring apparatus according to an exemplary embodiment of the present inventive concept.

As shown in FIG. 3, by using multiple ultrasound probes 10, a subject is inspected (operation 50). Each of the multiple ultrasound probes 10 may contact the subject from a different angle, similarly as illustrated in FIG. 2, and each may irradiate ultrasound toward the inside of the subject.

Each ultrasound probe 10 receives an ultrasound signal which is reflected from inner tissues of the subject and then returned, and each ultrasound probe 10 then converts the received signal into an electrical signal. Additionally, the corresponding sensor 15 mounted on each respective ultrasound probe 10 senses a relative position with respect to multiple ultrasound probes 10 when the subject is measured, thus producing position information (operation 51).

For instance, in the case where two ultrasound probes 10 are used for ultrasound measurement of a subject, a second sensor 15 mounted on a second ultrasound probe 10 may sense a position of the second ultrasound probe 10 relative to a first sensor 15 provided in a first ultrasound probe 10. In particular, the sensed position information may include information relating to how far the second ultrasound probe 10 is spaced apart from the first ultrasound probe 10, thus measuring the second ultrasound probe, and information as to how near a directional angle of the second ultrasound probe is to a desired angle with respect to the first ultrasound probe, more particularly, information relating to a distance and/or angle between the first and second ultrasonic probes.

The control part 20 may receive a plurality of electrical signals relating to a subject from the respective ultrasound probes 10 and then produce a corresponding plurality of image signals (operation 52).

When the plurality of image signals is produced, the control part 20 uses the position information relating to the multiple ultrasound probes 10, as sensed by the sensors 15 mounted on the ultrasound probes 10, to compensate for one or more errors relating to the respective image signals (operation 53). Because each of the plurality of image signals is a result of a measurement of the subject from a different relative angle and position, a particular image which is not obtained from one of the measured results may instead be exhibited from a different one of the measured results. Accordingly, in order to obtain desired results, the control part 20 compares a plurality of image signals using the position information relating to multiple ultrasound probes 10 sensed by the sensors 15. Then, by comparing the signals, an error relating to the respective image signals may be compensated for while removing an artifact.

The control part 20 may synthesize the compensated respective image signals to produce a synthesized image signal and display an image of the subject on the display (operation 54). By synthesizing the compensated respective image signals from which one or more artifacts has been removed, a synthesized image signal is produced, thus attaining more clear images of internal tissues of the subject.

Although exemplary embodiments of the present disclosure have been described above with reference to the accompanying drawings, it is clearly understood that these exemplary embodiments do not particularly restrict the scope of the present disclosure. Accordingly, it would be appreciated by those skilled in the art that various substitutions, variations and/or modifications may be made in these exemplary embodiments without departing from the principles and spirit of the disclosure. Therefore, it is understood that the scope of the present disclosure is defined not by the detailed description of the technical configurations and arrangements illustrated above, but by the appended claims, and all differences within the scope will be construed as being included in the present disclosure.

What is claimed is:

1. An ultrasound measuring apparatus, comprising:
a plurality of ultrasound probes having a sensor which detects a position of the corresponding probe with respect to each of the other ultrasound probes; and
a control part that uses a corresponding plurality of signals received from the plurality of ultrasound probes to generate a corresponding plurality of image signals relating to a subject toward which an ultrasound signal has been irradiated, and uses position information relating to the plurality of ultrasound probes, the position information being received from the sensor, to compensate for an error relating to the plurality of image signals.

2. The apparatus according to claim 1, wherein the sensor detects position information which includes a distance between the corresponding ultrasound probe and the other ultrasound probes in the plurality of ultrasound probes and an angular difference between a direction of the corresponding ultrasound probe with respect to the subject and a direction of the other ultrasound probes with respect to the subject.

3. The apparatus according to claim 1, wherein the position information is determined for the plurality of ultrasound probes with respect to the others of the plurality of ultrasound probes.

4. The apparatus according to claim 1, wherein the control part is configured to synthesize the plurality of image signals obtained after compensation for the error into a synthesized image signal.

5. A method for using ultrasound to generate a synthesized image signal, comprising:
receiving an ultrasound signal reflected from a subject from a plurality of ultrasound probes, the plurality of ultrasound probes having at least one sensor that can inspect a relative position of the corresponding ultrasound probe with respect to the others of the plurality of ultrasound probes;
generating a plurality of corresponding image signals by using the received signals from the plurality of ultrasound probes;
compensating for at least one error relating to the plurality of image signals to produce a corresponding plurality of compensated signals; and
synthesizing the compensated signals to produce the synthesized image signal.

6. The method according to claim 5, wherein the reception of an ultrasound signal from the plurality of ultrasound probes comprises inspecting the subject from different positions using the plurality of ultrasound probes, and detecting position information including an angular difference and/or a distance relating to a corresponding one of the plurality of ultrasound probes with respect to the others of the plurality of ultrasound probes, which position information is detected by the at least one sensor.

7. The method according to claim 6, wherein, for the plurality of ultrasound probes, positions of the others of the plurality of ultrasound probes are determined as the position information.

8. The method according to claim 5, wherein the generation of the plurality of image signals comprises inspecting the subject using the plurality of ultrasound probes from a different respective position to obtain a respective signal, and producing a plurality of image signals relating to the subject using the obtained respective signals.

9. The method according to claim 5, wherein the compensating for the at least one error comprises using position information relating to the plurality ultrasound probes which is detected by the at least one sensor to compensate for the at least one error relating to the plurality of image signals.

10. The method according to claim 9, wherein the compensating for the at least one error further comprises compensating for an artifact which occurs in at least one of the image signals as a result of a position of at least one of the plurality of ultrasound probes by using relative position information relating to the plurality of ultrasound probes which is detected by the at least one sensor.

11. An ultrasound measuring device, comprising:
a first ultrasound probe, including a first transducer which emits ultrasound signals and receives reflected ultrasound signals, and a first transceiver;
a second ultrasound probe, including a second transducer which emits ultrasound signals and receives reflected ultrasound signals, and a second transceiver;
at least one sensor to detect position information relating to the first ultrasound probe and the second ultrasound probe;
a controller in communication with each of the first and second transceivers; and
a display,
wherein the at least one sensor is configured to sense first position information relating to a position of the first ultrasound probe with respect to the second ultrasound probe and a subject, and sense second position information relating to a position of the second ultrasound probe with respect to the first ultrasound probe and the subject; and
wherein, when the first transducer receives a first reflected ultrasound signal, the first transducer is configured to convert the received first reflected ultrasound signal into a first electrical signal, and the first transceiver is configured to transmit the first electrical signal and the first position information to the controller; and
wherein, when the second transducer receives a second reflected ultrasound signal, the second transducer is configured to convert the received second reflected ultrasound signal into a second electrical signal, and the second transceiver is configured to transmit the second electrical signal and the second position information to the controller; and
wherein the controller is configured to use the received first and second electrical signals and the received first position information and the received second position information to generate a synthesized image signal; and
wherein the display is configured to use the synthesized image signal to display an image relating to the subject.

12. The device of claim 11, wherein the controller is further configured to use the received first position information and the received second information to determine an error relating to the first and second electrical signals and to compensate for the determined error prior to generating the synthesized image signal.

13. The device of claim 11, wherein the first position information includes a distance between the first ultrasound probe and the second ultrasound probe and an angular difference between a direction of the first ultrasound probe with respect to the subject and a direction of the second ultrasound probe with respect to the subject.

14. The device of claim 11, further comprising at least a third ultrasound probe, including a third transducer which emits ultrasound signals and receives reflected ultrasound signals, and a third transceiver,
wherein the at least one sensor is configured to sense third position information relating to a position of the third ultrasound probe with respect to each of the first and second ultrasound probes and the subject; and
wherein, when the third transducer receives a third reflected ultrasound signal, the third transducer is configured to convert the received third reflected ultrasound signal into a third electrical signal, and the third transceiver is configured to transmit the third electrical signal and the third position information to the controller; and
wherein the controller is further configured to use the received third electrical signal and the received third position information to generate the synthesized image signal.

15. A method of displaying an image relating to a subject by using ultrasound radiation, comprising:
irradiating first ultrasound toward the subject from a first position and second ultrasound toward the subject from a second position;
receiving a first reflected ultrasound signal at the first position and a second reflected ultrasound signal at the second position;
converting the first reflected ultrasound signal into a first electrical signal;
converting the second reflected ultrasound signal into a second electrical signal;
detecting position information relating to the first position with respect to the second position and the subject;
using the detected position information and the first and second electrical signals to generate a synthesized image signal; and
using the synthesized image signal to display the image relating to the subject.

16. The method of claim 15, further comprising using the detected position information to determine an error relating to the first and second electrical signals and to compensate for the determined error prior to generating the synthesized image signal.

17. The method of claim 15, wherein the detected position information includes a distance between the first position and the second position and an angular difference between a direction of the first position with respect to the subject and a direction of the second position with respect to the subject.

18. The method of claim 15, further comprising:
irradiating third ultrasound toward the subject from a third position;
receiving a third reflected ultrasound signal at the third position;
converting the third reflected ultrasound signal into a third electrical signal;
detecting additional position information relating to the third position with respect to the first and second positions and the subject; and
using the detected additional position information and the third electrical signal to generate the synthesized image signal.
